(19) United States
(12) Patent Application Publication
(10) Pub. No.: US 2003/0023228 A1
(43) Pub. Date: Jan. 30, 2003

(54) Ocular iontophoretic device and method for using the same

(76) Inventors: Thomas M. Parkinson, White Salmon, WA (US); Malgorzata Szlek, Salt Lake City, UT (US); Lindsay B. Lloyd, Salt Lake City, UT (US)

Correspondence Address: FACTOR & PARTNERS, LLC, 1327 W. WASHINGTON BLVD., SUITE 5G/H, CHICAGO, IL, 60607, US

(21) Appl. No.: 09/910,443

(22) Filed: Jul. 20, 2001

(51) Int. Cl. A61M 31/00

(52) U.S. Cl. 604/521; 604/20

(57) ABSTRACT

An ocular iontophoretic device for delivering a composition to an affected area of a living being's eye comprising an active electrode assembly associated with a matrix, wherein the matrix includes a composition capable of treating inflammatory and/or neovascularization conditions.

A method for treating an affected area of a living being's eye comprising the steps of: associating a composition with an ocular iontophoretic device; positioning at least a portion of the ocular iontophoretic device on the eye of a living being; and iontophoretically delivering the composition to an affected area of the living being's eye.

### BACKGROUND OF THE INVENTION

1. Field of the Invention

The present invention relates in general to an ocular iontophoretic device, and more particularly, to an ocular iontophoretic device which, upon association with the eye of a living being, and application of an electrical potential difference, iontophoretically delivers a composition containing a therapeutic steroid into the living being's eye, thereby treating directly or indirectly ocular inflammatory and/or conditions characterized by choroidal or retinal neovascularization.

2. Background Art

Certain adrenocortical steroids of the glucocortical class such as fludrocortisone, hydrocortisone, hydrocortisone acetate, hydrocortisone cypionate, hydrocortisone sodium phosphate, hydrocortisone sodium succinate, beclomethsone dipropionate, betamethasone, benzoate, betamethasone dipropionate, betamethasone sodium phosphate, betamethsone acetate, betamethasone valerate, cortisone acetate, dexamethasone, dexamethasone sodium phosphate, dexamethasone acetate, flunisolide, methylprenisolone, methylprenisolone acetate, methylprenisolone sodium succinate, paramethasone acetate, prednisolone, prednisolone acetate, prednisolone sodium phosphate, prednisolone tebutate, prednisone, triamcinolone, triamcinolone acetonide, triamcinolone diacetate, triamcinolone hexacetonide, alclometasone dipropionate, amcinonide, clobetasol propionate, clocortolone pivalate, cortisol butyrate, cortisol valerate, desonide, desoximetasone, diflorasone diacetate, fluocinonide, fluorometholone, flurandrenolide, halcinonide, medryosone, and mometasone furoate have been known in the art for years, and have been shown to possess anti-inflammatory and immunosuppresive activities. As such, several of these agents have been used to treat inflammatory conditions of the eye. Dexamethasone and betamethasone are of particular interest in treating anterior, intermediate, or posterior uveitis including specific forms such as Iridocyclitis, Behcet's Syndrome, sarcoidosis, and Vogt-Koyanagi-Harada Syndrome. Other inflammatory conditions including scleritis, cystoid macular edema, arteric anterior ischemic optic neuropathy have also been treated with these compounds. In addition, adrenocortical steroids have also been used as adjunct therapy for ocular conditions of endopthalmitis, Grave's ophthalmopathy, and tramatic optic neuropathies.

Recently, a number of investigators have evaluated the angiostatic activity of adrenocortical steroids because of the role inflammation may play in the initiation of ocular and other forms of angiogenisis. Several small clinical studies have suggested that either local or systemic administration of potent adrenocortical steroids such as dexamethasone or betamethasone can inhibit choroidal neovascularization (CNV). CNV can lead ultimately to loss of vision. This antiangiogenic activity, along with the anti-proliferative activity on fibroblasts suggests the potential utility of local administration of adrenocortical steroids to the eye to treat age related macular degeneration (AMD) and other ophthalmic conditions including ocular tumors which are characterized or facilitated by neovascularization.

Other steroids have also been shown to be useful in direct treatment of neovascularization of the structures within the eye. One of these is tetrahydrocortisol. Another is the Alcon compound Anecortave Acetate. Anecortave acetate, while being devoid of conventional glucocorticoid activity and associated side effects, completely inhibits ocular neovascularization in certain animal models and has been evaluated for use in treating AMD. Anecortave has also been evaluated for treating the condition pterygium which is a fibrovascular overgrowth of the cornea from the conjunctiva.

The aminosterols, which include Magainin Pharmaceutical's Squalamine, are another class of steroids that bind to and then enter endothelial and other activated cell types. This causes a decrease in cell motility, cytoskeletal activity, and cell adhesion, and also decreases cellular response to multiple growth factors thereby inhibiting angiogenesis, tumor proliferation and inflammation. Squalamine, in particular, has demonstrated anti-neovascular activity and as such may be useful in treating AMD and other ophthalmic conditions.

While administration of these various steroid compositions has been identified as a promising mechanism to remedy the above-identified conditions, delivery of a steroid composition to an affected area of a living being's eye remains largely problematic. Indeed, known prior art devices and associated methods of administering a steroid composition, identified hereinbelow, are replete with drawbacks and disadvantages.

For example, delivering an adrenocortical steroid of the glucocortical class to an affected, local area of a living being's eye for chronic conditions using a systemic delivery method is problematic because of the many well-known conventional side effects associated with chronic systemic delivery of adrenocortical steroids. Side effects include suppression of the pituitary-adrenal function, fluid and electrolyte disturbances, hypertension, hyperglycemia and glycosuria, increased susceptibility to infection, peptic ulcers, osteoporosis, myopathy, behaviorial disturbances, arrest of growth, and Cushing's habitus. In addition, rapid cessation of chronic systemic adrenocortical steroid treatment results in characteristic withdrawal syndrome consisting of fever, myalgia, arthragia, and malaise.

Systemic treatment of local ophthalmic chronic conditions of neovascularization with the non-glucocortical steroids (which minimize some of the listed side effects of the glucocortical steroids) also is not desirable because normal angiogenisis is required in other areas of the body for wound healing and prevention of conditions such as restenosis. Also CNS and cardiovascular toxicity problems have been observed with aminosterols.

Local delivery of therapeutic steroidal compositions to the eye has been attempted to avoid the systemic side effects of steroid delivery. Local delivery has been practiced via a variety of delivery modes including topical application and periocular, subconjunctival, or intervitreal injections. Topical formulations and injections have sometimes been of biodegradable polymeric compositions of a steroid to enhance persistence. Surgical implantation of biodegradable or non biodegradable polymeric controlled release devices has also been suggested to deliver steroids to the eye. These means of local steroidal delivery are also problematic.

Topical formulations are quickly washed away from the surface of the eye despite the use of polymeric formulations to increase their persistence. This requires that they be frequently applied. In some cases, drops are indicated as often as every fifteen minutes. Patient compliance is thus a problem, particularly at night. In addition, topical penetration of steroids into the eye tissue is generally poor so that therapeutic levels are difficult to achieve even in anterior regions of the eye. Topical formulations of steroids have not been demonstrated to be effective for treating conditions in the back of the eye.

Periocular injections are invasive, unpleasant, and penetration form the periocular region into the interior of the eye is again limited. Subconjuctival injections are also invasive. Both are demanding of the physician inasmuch as placement of the needle requires an extremely high level of precision.

Intravitreal injections do allow therapeutic levels of drug to be administered but can cause among other things, retinal detachment, bleeding into the interior of the eye, increased interocular pressure, and increased risk of secondary infection. Although perhaps justifiable for occasional acute conditions, these risk factors render interocular injection undesirable as a delivery mode for chronic administration. Furthermore, interocular injections can be scary, unpleasant, and painful for the patient.

Insertion of biodegradable controlled release devices requires surgery. Furthermore, as the device degrades, pieces of the device can break loose obstructing vision and causing sudden release of higher amounts of remaining steroid. Insertion of non degradable polymeric controlled release devices is invasive and requires either another surgery for explantation of the spent device or leaving the spent device behind in the interior of the eye.

### SUMMARY OF THE INVENTION

The present invention is directed to an ocular iontophoretic device for delivering a steroid composition to an affected area of a living being's eye comprising an active electrode assembly associated with a matrix, wherein the matrix includes a steroid composition capable of treating inflammatory and/or neovascularization conditions.

In preferred embodiments of the present invention, the matrix includes a composition consisting of an ionic, water soluble ester of dexamethasone, betamethasone, or anecortave steroid, including the 21 esters dexamethasone phosphate, betamethasone phosphate, or anecortave phosphate. Alternatively, the steroid in the matrix may be a water soluble salt of an amino sterol such as squalamine lactate or squalamine chloride.

In the preferred embodiment of the present invention, the steroid composition ranges in concentration from approximately 0.4 to approximately 4 weight percent in the aqueous portion of the hydrated matrix. In particular, a commercial formulation of dexamethasone sodium phosphate Inj., USP, equivalent to 10 mg/mL of dexamethasone phosphate active ingredient may be added to the dry matrix prior to a treatment. Each ml contains dexamethasone sodium phosphate equivalent to 10 mg dexamethasone phosphate, sodium sulfite anhydrous 1.5 mg, sodium citrate anhydrous 16.5 mg and benzyl alcohol 0.01 mL in water for injection. The pH is adjusted to 7.0-8.5; with sodium hydroxide and/or citric acid added, if needed. Other preferred embodiments of the present invention could include steroid compositions according to the above-defined concentrations and formulations with the use of an anecortave phosphate steroid or an amino steroid. Notwithstanding the above, and as would be readily understood to those having ordinary skill in the art, the above formulation and concentration are disclosed as a preferred embodiment. Other formulations and concentrations are also contemplated in accordance with the teachings of the present invention.

In yet other preferred embodiments of the present invention, the steroid compositions are formulated with tonicity from 0.5-1.8% sodium chloride equivalent but most preferably near isotonic and buffered to near pH 7.4 (slightly acid relative to this is better than being more basic than this. Formulation preservatives, such as antioxidants, antibacterials should be minimized by single dose packaging under nitrogen.

Preferably, the affected area of the eye is selected from at least one of the group consisting of the vitreous humor, cilliary body and iris, lens, cornea, optic disk, optic nerve, retina, choroid, circulation of the retina, circulation of the choroid, and sclera.

In accordance with the present invention, the ocular iontophoretic device further comprises a counter electrode assembly, wherein the counter electrode assembly is configured for completing an electrical circuit between the active electrode assembly and an energy source, and an energy source for generating an electrical potential difference.

The present invention is also directed to an ocular iontophoretic device for delivering a steroid composition to an affected area of a living being's eye, comprising: (a) a matrix, wherein the matrix is capable of temporarily retaining a solution having the steroid composition; (b) an active electrode assembly associated with the matrix, wherein the active electrode assembly is configured for iontophoretically delivering the steroid composition to the affected area of the living being's eye; (c) a counter electrode assembly, wherein the counter electrode assembly is configured for completing an electrical circuit between the active electrode assembly and an energy source; and (d) an energy source for generating an electrical potential difference. It is also contemplated that the ocular iontophoretic device comprises a reservoir, wherein the reservoir includes a steroid composition capable of treating inflammatory and/or neovascularization conditions.

The present invention is further directed to a method for treating an affected area of a living being's eye comprising the steps of: (a) associating a steroid composition with an ocular iontophoretic device; (b) positioning at least a portion of the ocular iontophoretic device on the eye of a living being; and (c) iontophoretically delivering the steroid composition to an affected area of the living being's eye.

In a preferred embodiment of the present invention, the step of associating the steroid composition includes the step of associating a steroid capable of treating inflammatory and/or neovascularization conditions.

In another preferred embodiment of the present invention, the step of iontophoretically delivering the steroid composition includes the step of iontophoretically delivering the steroid composition to at least one of the group consisting of the vitreous humor, cilliary body and iris, lens, cornea, optic disk, optic nerve, retina, choroid, circulation of the retina, circulation of the choroid, conjuctiva and sclera

In yet another preferred embodiment of the present invention, the step of iontophoretically delivering the steroid composition includes the step of iontophoretically loading a sclera of the living being's eye with the steroid composition for prolonged delivery into back regions of the living being's eye.

Preferably, the step of iontophoretically delivering the steroid composition, includes the step of iontophoretically delivering the steroid composition at a current between approximately 0.5 mA and approximately 4 mA for a period of between approximately 5 and approximately 60 minutes.

In accordance with the present invention, the step of iontophoretically delivering the steroid composition includes the step of delivering the steroid composition using negative polarity electrical current.

In a preferred embodiment of the present invention, the step of positioning at least a portion of the ocular iontophoretic device on the eye of a living being includes the step of applying at least a portion of the ocular iontophoretic device to a conjunctival surface in a region of a pars planum and insertions of an anterior cilliary artery.

The present invention is further directed to a method for treating inflammatory and/or neovascularization conditions within an affected area of a living being's eye comprising the steps of: (a) associating a steroid composition with a matrix of an ocular iontophoretic device; (b) associating the ocular iontophoretic device having an active electrode assembly with the eye of the living being; (c) iontophoretically delivering an effective amount of the steroid composition to an affected area of the living being's eye having a inflammatory and/or neovascularization condition; and (d) treating the affected area of the living being's eye, and, in turn, reducing or eliminating effects of the inflammatory and/or neovascularization condition.

### BRIEF DESCRIPTION OF THE DRAWINGS

The invention will now be described with reference to the drawings wherein:

FIG. 1 of the drawings is a cross-sectional schematic representation of a first embodiment of an ocular iontophoretic device fabricated in accordance with the present invention;

FIG. 2 of the drawings is a cross-sectional schematic representation of a first embodiment of an ocular iontophoretic device fabricated in accordance with the present invention showing the association of a counter electrode assembly and an energy source; and

FIG. 3 of the drawings is a cross-sectional schematic representation of a second embodiment of an ocular iontophoretic device fabricated in accordance with the present invention.

### DETAILED DESCRIPTION OF THE INVENTION

While this invention is susceptible of embodiment in many different forms, there is shown in the drawings and will herein be described in detail several specific embodiments with the understanding that the present disclosure is to be considered as an exemplification of the principles of the invention and is not intended to limit the invention to the embodiments illustrated.

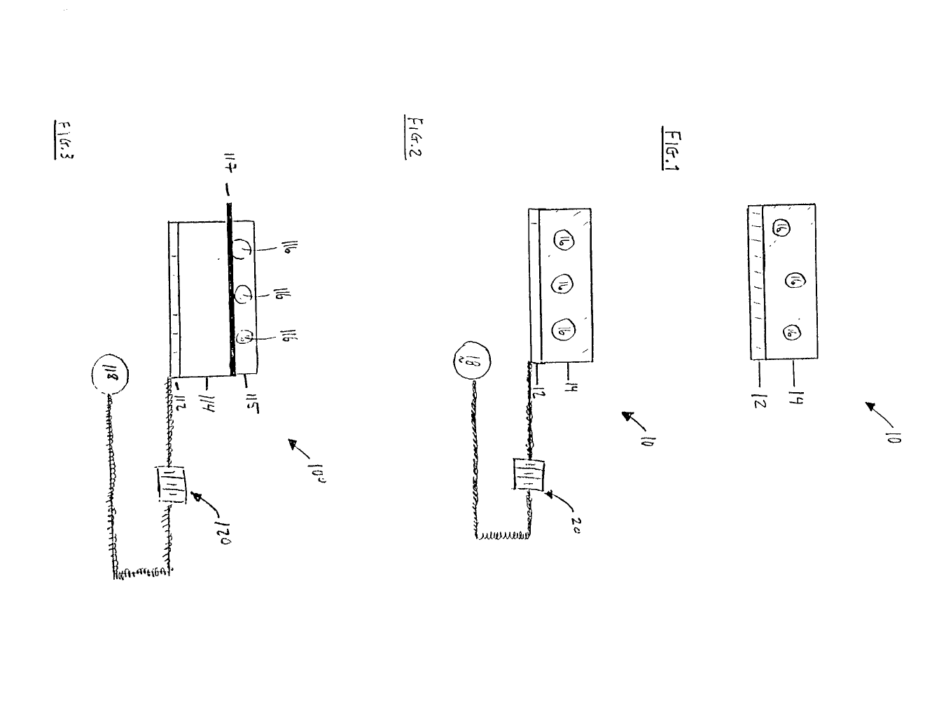

Referring now to the drawings and to FIG. 1 in particular, a first embodiment of an ocular iontophoretic device 10 is shown, which generally comprises active electrode assembly 12 and matrix 14. It will be understood that FIG. 1 is merely a cross-sectional schematic representation of ocular iontophoretic device 10. As such, some of the components have been distorted from their actual scale for pictorial clarity. As will be discussed in greater detail below, ocular iontophoretic device 10 is configured for delivering a steroid composition to an affected area of a living being's eye, thereby treating inflammatory and/or neovascularization conditions. By iontophoretically administering a steroid composition to the affected area of the eye, diseases associated with, among other things, inflammatory and/or neovascularization conditions can be efficiently remedied—especially including diseases of the eye wherein the affected area is toward the back of the eye, or generally proximate the optic nerve. Ocular iontophoretic device 10 offers many advantages over the previously discussed prior art devices and associated methods, including, but not limited to, simultaneous enablement of non-invasive and deep steroid delivery, non-invasive local delivery of an effective, therapeutic level of steroid while minimizing systemic concentrations, and enablement of sclera loading for prolonged delivery into regions in the front or back of the eye.

Active electrode assembly 12 generally comprises a conductive material, which upon application of an electrical potential difference thereto, drives an ionic steroid composition (i.e. an ionic medicament), received from matrix 14 and delivers the steroid composition into predetermined tissues and surrounding structures of a living being. It will be understood that active electrode assembly 12 may comprise an anode (for aminosterols like squalamine lactate) or a cathode anode (for phosphate esters of steroids) depending upon whether the medicament is cationic or anionic in form. It will be further understood that active electrode assembly may include an open-faced or high current density electrode. As would be readily understood to those having ordinary skill in the art, any one of a number of conventional active electrode assemblies are contemplated for use in accordance with the present invention. The only contemplated limitation relative to active electrode assembly 12 is that it must be geometrically and compositionally compatible for ocular applications of living beings, most relevantly, humans.

Matrix 14 extends contiguously from active electrode 12, and is preferably fabricated from a material capable of temporarily retaining ionic steroid composition 16 in solution. The solution may also contain supplemental agents, such as electrolytes, stability additives, medicament preserving additives, pH regulating buffers, etc. Matrix 14 may comprise, for example, a natural or synthetic amorphous member, a natural or synthetic sponge pad, a natural or synthetic lint free pad, a natural or synthetic low particulate member—just to name a few. Indeed, numerous other materials that would be known to those having ordinary skill in the art having the present disclosure before them are likewise contemplated for use. As with active electrode assembly 12, the only contemplated limitation relative to matrix 14 is that it must be geometrically and compositionally compatible for ocular applications of living beings, most relevantly, humans.

Steroid composition 16 is retained within matrix 14. In accordance with the present invention, ionic medicament 16 comprises a dexamethasone composition which is capable of treating inflammatory and/or neovascularization conditions. Such a dexamethasone composition ranges in molecular weight ranging from approximately 400 to approximately 600. Preferred dexamethasone composition includes inorganic esters of dexamethasone, such as 9-fluoro-11β,17-dihydroxy-16α-methyl-21-(phosphonooxy)pregna-1,4-diene-3,20-dione disodium salt—a dexamethasone sodium phosphate salt. Examples of suitable dexamethasone compositions for use in accordance with the present invention include dexamethasone sodium phosphate Inj., USP, equivalent to 10 mg/mL of dexamethasone phosphate. available from Elkins-Sinn, Inc. of Cherry Hill, N.J. It will be understood that other dexamethasone compositions that would be known to those having ordinary skill in the art having the present disclosure before them are likewise contemplated for use. It will also be understood that other steroids, such as those within the classes of anecortave phosphate steroids and amino steroles, that would be known to those having ordinary skill in the art having the present disclosure before them are likewise contemplated for use.

As is shown in FIG. 2, ocular iontophoretic device 10 may also include counter electrode assembly 18 and energy source 20. Counter electrode assembly 18 may be housed within ocular iontophoretic device 10, or alternatively, may be remotely associated with ocular iontophoretic device 10 via conventional electrical conduit. Counter electrode assembly 18 is configured for completing an electrical circuit between active electrode assembly 12 and an energy source 20. As with active electrode 12, counter electrode 18 may comprise an anode or a cathode depending upon whether the medicament is cationic or anionic in form. As would be readily understood to those having ordinary skill in the art, any one of a number of counter electrodes are contemplated for use in accordance with the present invention.

Similarly to counter electrode assembly 18, energy source 20 may be housed within ocular iontophoretic device 10, or alternatively, may be remotely associated with ocular iontophoretic device 10 via conventional electrical conduit. Energy source 20 preferably supplies low voltage constant direct current between approximately 0.5 milliamps (mA) and approximately 4 mA for generating an electrical potential difference. The energy source may also provide for an initial higher voltage during current ramp-up to break down higher initial tissue resistance as in commercial power supply units used for transdermal iontophoresis. For purposes of the present disclosure, energy source 20 may include one or more primary or secondary electrochemical cells. While specific examples of energy source 20 have been disclosed, for illustrative purposes only, it will be understood that other energy sources known to those having ordinary skill in the art having the present disclosure before them are likewise contemplated for use.

Referring now to the drawings and to FIG. 3 in particular, a second embodiment of an ocular iontophoretic device 100 is shown, which generally comprises active electrode assembly 112, matrix 114, reservoir 115, counter electrode assembly 118, and energy source 120. It will be understood that active electrode assembly 112, matrix 114, counter electrode assembly 118, and energy source 120, are configured analogously to previously discussed active electrode assembly 12, matrix 14, counter electrode assembly 18, and energy source 20, respectively. Ocular iontophoretic device 100 is configured for delivering a dexamethasone composition to an affected area of a living being's eye for treating inflammatory and/or neovascularization conditions therein.

Reservoir 115 includes steroid composition 116, in solution, which is capable of treating inflammatory and/or neovascularization conditions. Reservoir 115 may include a releasable cover member 117 which, upon articulation, releases steroid composition 116 into matrix 114. Such a release cover enables prompt delivery of the steroid composition with very little device preparation.

The present invention is also directed to a method for treating an affected area of a living being's eye comprising the following steps. First, a steroid composition is associated with an ocular iontophoretic device. Preferably the steroid composition is metered from a syringe or single unit dose. Second, at least a portion of the ocular iontophoretic device is positioned on the eye of a living being. Finally, the steroid composition is iontophoretically delivered to an affected area of the living being's eye. Preferably, the delivery lasts for between approximately 5 and approximately 20 minutes. Compared to prior art administration or delivery methods, the present invention enables a generally painless, non-invasive and deep delivery of the steroid composition. Moreover, the steroid composition is locally delivered to an affected area of a living being's eye at an effective, therapeutic level. Preferred ocular delivery regions include the vitreous humor, optic disk, optic nerve, retina, choroid, circulation of the retina, circulation of the choroid, and sclera. It is likewise contemplated that delivery to front regions of the eye (including aqueous humor, ciliary body, iris, and lens) may be administered.

The present invention is also directed to a method for treating inflammatory and/or neovascularization conditions within an affected area of a living being's eye comprising the following steps. First, a steroid composition is associated with the matrix of the ocular iontophoretic device. Second, an effective amount of the steroid composition is iontophoretically delivered to an affected area of the living being's eye. Third, the affected area is treated, thereby reducing or eliminating the effects of an inflammatory and/or neovascularization condition within an affected area of a living being's eye.

The foregoing description merely explains and illustrates the invention and the invention is not limited thereto except insofar as the appended claims are so limited, as those skilled in the art who have the disclosure before them will be able to make modifications without departing the scope of the invention.

What is claimed is:

1. An ocular iontophoretic device for delivering a steroid composition to an affected area of a living being's eye, comprising:
an active electrode assembly associated with a matrix, wherein the matrix includes a dexamethasone composition capable of treating inflammatory and/or neovascularization conditions.

2. The ocular iontophoretic device according to claim 1, wherein the matrix includes an ionic ester of a steroid.

3. The ocular iontophoretic device according to claim 1, wherein the matrix includes dexamethasone sodium phosphate.

4. The ocular iontophoretic device according to claim 1, wherein the matrix includes 9 fluoro-11β,17-dihydroxy-16α-methyl-21-(phosphonooxy)pregna-1,4-diene-3,20-dione disodium salt.

5. The ocular iontophoretic device according to claim 3, wherein the dexamethasone composition ranges in molecular weight from approximately 400 to approximately 600.

6. The ocular iontophoretic device according to claim 3, wherein the dexamethasone composition is formulated in an approximately 0.5% by weight to approximately 4% by weight compound, and preferably 1% by weight.

7. The ocular iontophoretic device according to claim 6, wherein the buffer ranges in pH from approximately 6 to approximately 8.5, and preferably 7.4.

8. The ocular iontophoretic device according to claim 1, wherein the buffer ranges in pH from approximately 6 to approximately 8.5, and preferably 7.4.

9. The ocular iontophoretic device according to claim 1, wherein the affected area of the eye is selected from at least one of the group consisting of the vitreous humor, retina, choroid, circulation of the retina, circulation of the choroid, and sclera.

10. The ocular iontophoretic device according to claim 1, further comprising:
a counter electrode assembly, wherein the counter electrode assembly is configured for completing an electrical circuit between the active electrode assembly and an energy source; and
an energy source for generating an electrical potential difference.

11. The ocular iontophoretic device according to claim 1, wherein the active electrode assembly includes an open-faced or high current density electrode.

12. An ocular iontophoretic device for delivering a dexamethasone composition to an affected area of a living being's eye, comprising:
a matrix, wherein the matrix is capable of temporarily retaining a solution having the dexamethasone composition;
an active electrode assembly associated with the matrix, wherein the active electrode assembly is configured for iontophoretically delivering the dexamethasone composition to the affected area of the living being's eye;
a counter electrode assembly, wherein the counter electrode assembly is configured for completing an electrical circuit between the active electrode assembly and an energy source; and
an energy source for generating an electrical potential difference.

13. The ocular iontophoretic device according to claim 12, wherein the matrix includes an inorganic ester of dexamethasone.

14. The ocular iontophoretic device according to claim 12, wherein the matrix includes a dexamethasone sodium phosphate.

15. The ocular iontophoretic device according to claim 12, wherein the matrix includes 9 fluoro-11β,17-dihydroxy-16α-methyl-21-(phosphonooxy)pregna-1,4-diene-3,20-dione disodium salt.

16. The ocular iontophoretic device according to claim 12, wherein the dexamethasone composition ranges in molecular weight from approximately 400 to approximately 600.

17. The ocular iontophoretic device according to claim 12, wherein the dexamethasone composition is formulated in an approximately 0.5% by weight to approximately 4% by weight compound, and preferably 1% by weight.

18. The ocular iontophoretic device according to claim 17, wherein the buffer ranges in pH from approximately 6 to approximately 8.5, and preferably 7.4.

19. The ocular iontophoretic device according to claim 12, wherein the buffer ranges in pH from approximately 6 to approximately 8.5, and preferably 7.4.

20. The ocular iontophoretic device according to claim 12, wherein the affected area of the eye is selected from at least one of the group consisting of the vitreous humor, retina, choroid, circulation of the retina, circulation of the choroid, and sclera.

21. The ocular iontophoretic device according to claim 12, wherein the active electrode assembly includes an open-faced or high current density electrode.

22. An ocular iontophoretic device for delivering a dexamethasone composition to an affected area of a living being's eye, comprising:
a reservoir, wherein the reservoir includes a dexamethasone composition capable of treating inflammatory and/or neovascularization conditions;
a matrix, wherein the matrix is capable of temporarily retaining a solution having a dexamethasone composition;
an active electrode assembly associated with the matrix, wherein the active electrode assembly is configured for iontophoretically delivering the dexamethasone composition to the affected area of the living being's eye;
a counter electrode assembly, wherein the counter electrode assembly is configured for completing an electrical circuit between the active electrode assembly and an energy source; and
an energy source for generating an electrical potential difference.

23. A method for treating an affected area of a living being's eye, comprising the steps of:
associating a dexamethasone composition with an ocular iontophoretic device;
positioning at least a portion of the ocular iontophoretic device on the eye of a living being; and
iontophoretically delivering the dexamethasone composition to an affected area of the living being's eye.

24. The method according to claim 23, wherein the step of associating the dexamethasone composition includes the step of associating a dexamethasone composition capable of treating inflammatory and/or neovascularization conditions.

25. The method according to claim 23, wherein the step of iontophoretically delivering the dexamethasone composition includes the step of iontophoretically delivering the dexamethasone composition to at least one of the group consisting of the vitreous humor, retina, choroid, circulation of the retina, circulation of the choroid, and sclera.

26. The method according to claim 23, wherein the step of iontophoretically delivering the dexamethasone composition includes the step of iontophoretically loading a sclera of the living being's eye with the dexamethasone composition for prolonged delivery into back regions of the living being's eye.

27. The method according to claim 23, wherein the step of iontophoretically delivering the dexamethasone composition includes the step of iontophoretically delivering the dexamethasone composition at a current between approximately 0.5 mA and approximately 4 mA for a period of between approximately 5 and approximately 20 minutes.

28. The method according to claim 23, wherein the step of iontophoretically delivering the dexamethasone composition includes the step of delivering the dexamethasone composition using negative polarity electrical current.

29. The method according to claim 23, wherein the step of positioning at least a portion of the ocular iontophoretic device on the eye of a living being includes the step of applying at least a portion of the ocular iontophoretic device to a conjunctival surface in a region of a pars planum and/or insertions of an anterior cilliary artery.

30. A method for treating an inflammatory and/or neovascularization condition within an affected area of a living being's eye, comprising the steps of:
associating a dexamethasone composition with a matrix of an ocular iontophoretic device;
associating the ocular iontophoretic device having an active electrode assembly with the eye of the living being;
iontophoretically delivering an effective amount of the dexamethasone composition to an affected area of the living being's eye having an inflammatory and/or neovascularization condition; and
treating the affected area of the living being's eye, and, in turn, reducing or eliminating effects of the inflammatory and/or neovascularization condition.

31. The method according to claim 30, wherein the step of associating the dexamethasone composition includes the step of associating a dexamethasone composition capable of treating inflammatory and/or neovascularization conditions.

32. The method according to claim 30, wherein the step of iontophoretically delivering the dexamethasone composition includes the step of iontophoretically delivering the dexamethasone composition to at least one of the group consisting of the vitreous humor, retina, choroid, circulation of the retina, circulation of the choroid, and sclera.

33. The method according to claim 30, wherein the step of iontophoretically delivering the dexamethasone composition includes the step of iontophoretically loading a sclera of the living being's eye with the dexamethasone composition for prolonged delivery into back regions of the living being's eye.

34. The method according to claim 30, wherein the step of iontophoretically delivering the dexamethasone composition includes the step of iontophoretically delivering the dexamethasone composition at a current between approximately 0.5 mA and approximately 4 mA for a period of between approximately 5 and approximately 20 minutes.

35. The method according to claim 30, wherein the step of iontophoretically delivering the dexamethasone composition includes the step of delivering the dexamethasone composition using negative polarity electrical current.

36. The method according to claim 30, wherein the step of associating the ocular iontophoretic device having an active electrode assembly with the eye of the living being includes the step of applying at least a portion of the ocular iontophoretic device to a conjunctival surface in a region of a pars planum and/or insertions of an anterior cilliary artery.

37. An ocular iontophoretic device for delivering a steroid composition to an affected area of a living being's eye, comprising:
an active electrode assembly associated with a matrix, wherein the matrix includes a composition selected from the group consisting of corticosteroids, anecortave phosphate steroids, and amino steroles, capable of treating inflammatory and/or neovascularization conditions.

38. The ocular iontophoretic device according to claim 37, wherein the composition ranges in molecular weight from approximately 400 to approximately 600.

39. The ocular iontophoretic device according to claim 37, wherein the composition is formulated in an approximately 0.5% by weight to approximately 4% by weight compound, and preferably 1% by weight.

40. The ocular iontophoretic device according to claim 39, wherein the buffer ranges in pH from approximately 6 to approximately 8.5, and preferably 7.4.

41. The ocular iontophoretic device according to claim 37, wherein the buffer ranges in pH from approximately 6 to approximately 8.5, and preferably 7.4.

42. The ocular iontophoretic device according to claim 37, wherein the affected area of the eye is selected from at least one of the group consisting of the vitreous humor, retina, choroid, circulation of the retina, circulation of the choroid, and sclera.

43. An ocular iontophoretic device for delivering a composition to an affected area of a living being's eye, comprising:
a matrix, wherein the matrix is capable of temporarily retaining a solution having a composition selected from the group consisting of corticosteroids, anecortave phosphate steroids, and amino steroles, capable of treating inflammatory and/or neovascularization conditions;
an active electrode assembly associated with the matrix, wherein the active electrode assembly is configured for iontophoretically delivering the composition to the affected area of the living being's eye;
a counter electrode assembly, wherein the counter electrode assembly is configured for completing an electrical circuit between the active electrode assembly and an energy source; and
an energy source for generating an electrical potential difference.

44. The ocular iontophoretic device according to claim 43, wherein the composition ranges in molecular weight from approximately 400 to approximately 600.

45. The ocular iontophoretic device according to claim 43, wherein the composition is formulated in an approximately 0.5% by weight to approximately 4% by weight compound, and preferably 1% by weight.

46. The ocular iontophoretic device according to claim 45, wherein the buffer ranges in pH from approximately 6 to approximately 8.5, and preferably 7.4.

47. The ocular iontophoretic device according to claim 43, wherein the buffer ranges in pH from approximately 6 to approximately 8.5, and preferably 7.4.

48. The ocular iontophoretic device according to claim 43, wherein the affected area of the eye is selected from at least one of the group consisting of the vitreous humor, retina, choroid, circulation of the retina, circulation of the choroid, and sclera.

49. An ocular iontophoretic device for delivering a composition to an affected area of a living being's eye, comprising:
a reservoir, wherein the reservoir includes a composition capable of treating inflammatory and/or neovascularization conditions;
a matrix, wherein the matrix is capable of temporarily retaining a solution having a composition, wherein the composition is selected from the group consisting of corticosteroids, anecortave phosphate steroids, and amino steroles;
an active electrode assembly associated with the matrix, wherein the active electrode assembly is configured for iontophoretically delivering the composition to the affected area of the living being's eye;
a counter electrode assembly, wherein the counter electrode assembly is configured for completing an electrical circuit between the active electrode assembly and an energy source; and
an energy source for generating an electrical potential difference.

50. A method for treating an affected area of a living being's eye, comprising the steps of:
associating a composition with an ocular iontophoretic device, wherein the composition is selected from the group consisting of corticosteroids, anecortave phosphate steroids, and amino steroles;
positioning at least a portion of the ocular iontophoretic device on the eye of a living being; and
iontophoretically delivering the composition to an affected area of the living being's eye.

51. The method according to claim 50, wherein the step of associating the composition includes the step of associating a composition capable of treating inflammatory and/or neovascularization conditions.

52. The method according to claim 50, wherein the step of iontophoretically delivering the composition includes the step of iontophoretically delivering the composition to at least one of the group consisting of the vitreous humor, retina, choroid, circulation of the retina, circulation of the choroid, and sclera.

53. The method according to claim 50, wherein the step of iontophoretically delivering the composition includes the step of iontophoretically loading a sclera of the living being's eye with the composition for prolonged delivery into back regions of the living being's eye.

54. The method according to claim 50, wherein the step of iontophoretically delivering the composition includes the step of iontophoretically delivering the composition at a current between approximately 0.5 mA and approximately 4 mA for a period of between approximately 5 and approximately 20 minutes.

55. The method according to claim 50, wherein the step of iontophoretically delivering the composition includes the step of delivering the composition using negative polarity electrical current.

56. The method according to claim 50, wherein the step of positioning at least a portion of the ocular iontophoretic device on the eye of a living being includes the step of applying at least a portion of the ocular iontophoretic device to a conjunctival surface in a region of a pars planum and/or insertions of an anterior cilliary artery.

57. A method for achieving an effect of alleviating an inflammatory and/or neovascularization condition within an affected area of a living being's eye comprising the steps of:
associating a dexamethasone composition with a matrix of an ocular iontophoretic devide;
associating the ocular iontophoretic device having an active electrode assembly with the eye of the living being;
iontophoretically delivering an effective amount of the dexamethasone composition to an affected area of the living being's eye having an inflammatory and/or neovascularization condition; and
treating the affected area of the living being's eye, and, in turn, reducing or eliminating effects of the inflammatory and/or neovascularization condition.

58. A method for achieving an effect of alleviating an inflammatory and/or neovascularization condition within an affected area of a living being's eye comprising the steps of:
associating a composition with a matrix of an ocular iontophoretic devide, wherein the composition is selected from the group consisting of corticosteroids, anecortave phosphate steroids, and amino steroles;
associating the ocular iontophoretic device having an active electrode assembly with the eye of the living being;
iontophoretically delivering an effective amount of the composition to an affected area of the living being's eye having an inflammatory and/or neovascularization condition; and
treating the affected area of the living being's eye, and, in turn, reducing or eliminating effects of the inflammatory and/or neovascularization condition.
